(19) United States
(12) Patent Application Publication
(10) Pub. No.: US 2007/0167761 A1
(43) Pub. Date: Jul. 19, 2007

(54) ULTRASONIC DIAGNOSTIC APPARATUS

(76) Inventor: Hiroshi Hashimoto, Tokyo (JP)

Correspondence Address: Patrick W. Rasche;Armstrong Teasdale LLP, Suite 2600, One Metropolitan Square, St. Louis, MO, 63102, US

(21) Appl. No.: 11/566,413

(22) Filed: Dec. 4, 2006

(30) Foreign Application Priority Data
Dec. 7, 2005 (JP) 2005-354119

(51) Int. Cl. A61B 8/00

(52) U.S. Cl. 600/437

(57) ABSTRACT

This invention realizes high diagnosis efficiency in ultrasonic diagnosis. A start frame selecting unit selects a start frame corresponding to the time a display unit starts displaying slice images in chronological order, in slice images in a plurality of frames generated in chronological order by an image generating unit. The start frame selecting unit selects the start frame in accordance with time point when an injection time counting unit starts counting the time of injecting a contrast medium into the subject. The display unit sequentially displays the slice image of the start frame selected by the start frame selecting unit and the subsequent slice images of the frames generated in chronological order by the image generating unit.

### BACKGROUND OF THE INVENTION

The present invention relates to an ultrasonic diagnostic apparatus and, more particularly, to an ultrasonic diagnostic apparatus for generating slice images of a subject in a plurality of frames in chronological order on the basis of echo signals obtained by performing a scan of transmitting ultrasonic waves to the subject into which a contrast medium is injected and receiving the ultrasonic waves reflected from the subject to which the ultrasonic waves were transmitted, and sequentially displaying the slice images in the plurality of frames.

The ultrasonic diagnostic apparatus generates, for example, an image of a slice of a subject on the basis of echo signals obtained by performing a scan of transmitting ultrasonic waves to the subject and receiving the ultrasonic waves reflected from the subject to which the ultrasonic waves have been transmitted, and displays the image on a display screen. Since the ultrasonic diagnostic apparatus can easily acquire images of a slice of a subject in a real-time manner, particularly, it is often used in the medical field for fetal medical check, heart check, and the like.

The ultrasonic diagnostic apparatus has various display modes such as B mode (Brightness mode), M mode (Motion mode), and Doppler mode. The B mode is a mode of displaying an image obtained by converting changes in intensity of ultrasonic echoes from a subject into changes in brightness, and is used at the time of, for example, obtaining an image of a slice of a subject. The M mode is a mode of displaying, in chronological order, brightness in parts each corresponding to one sound ray of an ultrasonic echo in a plurality of B mode images which are sequentially displayed in chronological order. For example, the M mode is used to obtain an image of the motion of a moving organ such as the heart valve in a subject. The Doppler mode is a mode using the Doppler effect that the frequency of an ultrasonic echo reflected from a moving body shifts in proportion to moving velocity of the moving body and is used for obtaining an image of blood current information such as moving velocity of blood flowing in a subject.

The ultrasonic diagnostic apparatus has a primary storage such as a cine memory and a secondary storage such as an HDD (Hard Disc Drive). The cine memory temporarily stores data of slice images in a plurality of frames continuously obtained in chronological order by a scan. Data of a moving picture made by the slice images in the plurality of frames is output from the cine memory and stored in the HDD (refer to, for example, Patent Document 1).

[Patent Document 1] Japanese Patent Laid-Open No. 2002-112254

Data of a moving picture stored in a storage such as a cine memory is reproduced on a display screen and used after a scan is stopped. For example, data is sequentially displayed in chronological order from the slice image of the first frame stored in the cine memory.

Consequently, in the case of starting display from slice images in frames in a necessary range in stored moving pictures, the operator has to repeat operations of rewinding and rapid traverse of frames by using an operation device.

Since the complicated operations are necessary, it is difficult to realize high diagnosis efficiency.

In particular, when image acquisition time is long like in the case of obtaining an image of a subject by using a contrast medium, the inconvenience is conspicuous since a moving picture made of slice images of a number of frames is stored.

At the time of displaying a plurality of moving pictures side by side on a display screen, they are sequentially displayed from the first frame of each of the moving pictures. Consequently, in the case where the position in time series of frames in a necessary range various among the moving pictures, since it is difficult to synchronously display images of the frames in the necessary range in the moving pictures, it is not easy to conduct a diagnosis by comparing the plurality of moving pictures arranged on the display screen. There is a case such that, since it is difficult to display moving pictures synchronously with the timing of injecting a contrast medium into a subject, the inconvenience is conspicuous.

### SUMMARY OF THE INVENTION

Therefore, an object of the present invention is to provide an ultrasonic diagnostic apparatus realizing reduction in complicated operations and improved diagnostic efficiency.

To achieve the object, the present invention provides an ultrasonic diagnostic apparatus including: a scanning unit for obtaining echo signals by performing a scan of transmitting ultrasonic waves to a subject into which a contrast medium is injected and receiving the ultrasonic waves reflected from the subject to which the ultrasonic waves were transmitted; an image generating unit for generating slice images of the subject in a plurality of frames in chronological order on the basis of the echo signals obtained by the scanning unit; a storing unit for storing the slice images in the plurality of frames generated by the image generating unit; and a display unit for sequentially displaying the slice images in the plurality of frames stored in the storing unit so as to correspond to the chronological order in which the image generating unit has generated the plurality of frames. The apparatus includes: a start frame selecting unit for selecting a start frame at the time the display unit starts displaying the slice images in the chronological order from the slice images in the plurality of frames generated in the chronological order by the image generating unit; and an injection time counting unit for counting time in which the contrast medium is injected into the subject. The start frame selecting unit selects, as the start frame, a frame generated by the image generating unit when the injection time counting unit starts counting the time in which the contrast medium is injected into the subject, and the display unit sequentially displays the slice image of the start frame selected by the start frame selecting unit and the subsequent slice images in the frames generated in the chronological order by the image generating unit.

According to the present invention, an ultrasonic diagnostic apparatus realizing reduction in complicated operations and improvement in diagnostic efficiency can be provided.

Further objects and advantages of the present invention will be apparent from the following description of the preferred embodiments of the invention as illustrated in the accompanying drawings.

### BRIEF DESCRIPTION OF THE DRAWINGS

FIG. 1 is a block diagram showing the configuration of an ultrasonic diagnostic apparatus 1 in a first embodiment of the present invention.

FIG. 2 is a flowchart showing operations performed at the time of imaging a subject in the first embodiment of the invention.

FIG. 3 is a diagram showing a state where a start frame selecting unit 326 selects a start frame at the time of starting display in chronological order in the first embodiment of the invention.

FIG. 4 is a block diagram showing an operation unit 325 in a second embodiment of the invention.

FIG. 5 is a diagram showing a state where the start frame selecting unit 326 selects a start frame at the time of starting display in chronological order in the second embodiment of the invention.

FIG. 6 is a block diagram showing an image generating unit 322 and the operation unit 325 in a third embodiment of the invention.

FIG. 7 is a diagram showing a start frame position image generated by a start frame position image generating unit 322a in the third embodiment of the invention.

FIG. 8 is a diagram showing a state where the start frame selecting unit 326 selects a start frame at the time of starting display in chronological order in the third embodiment of the invention.

FIG. 9 is a block diagram showing the configuration of an ultrasonic diagnostic apparatus 1 a in a fourth embodiment of the invention.

FIG. 10 is a flowchart showing operations performed at the time of imaging a subject in the fourth embodiment of the invention.

FIG. 11 is a diagram showing a state where an end frame selecting unit 328 selects a start frame at the time of finishing display in chronological order in the fourth embodiment of the invention.

FIG. 12 is a block diagram showing the operation unit 325 in a fifth embodiment of the invention.

FIG. 13 is a diagram showing a state where the end frame selecting unit 328 selects an end frame at the time of finishing display in chronological order in the fifth embodiment of the invention.

FIG. 14 is a block diagram showing the image generating unit 322 and the operation unit 325 in a sixth embodiment of the invention.

FIG. 15 is a diagram showing an end frame position image generated by an end frame position image generating unit 322b in the sixth embodiment of the invention.

FIG. 16 is a diagram showing a state where the end frame selecting unit 328 selects an end frame at the time of finishing display in chronological order in the sixth embodiment of the invention.

FIG. 17 is a diagram showing a state where a display unit 41 displays a first moving picture MP1 and a second moving picture MP2 side by side on a display screen in a seventh embodiment of the invention.

### DETAILED DESCRIPTION OF THE INVENTION

Embodiments of the present invention will be described hereinbelow.

### First Embodiment

A first embodiment of the invention will be described.

Apparatus Configuration

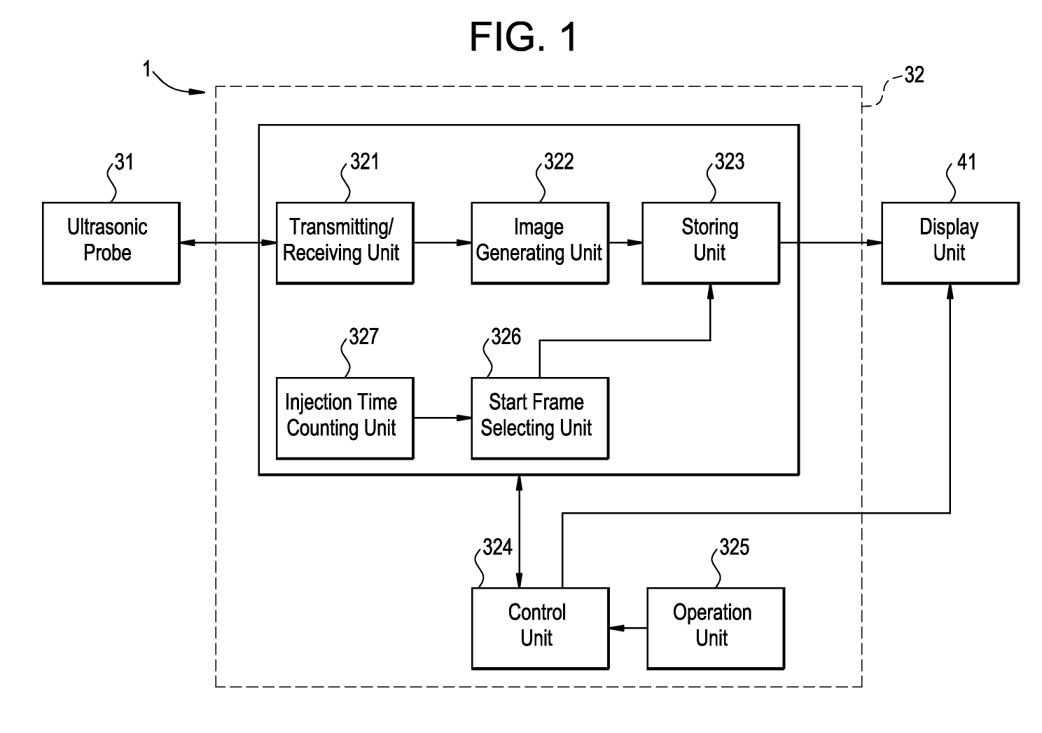

FIG. 1 is a block diagram showing the configuration of an ultrasonic diagnostic apparatus 1 in the first embodiment of the invention.

As shown in FIG. 1, the ultrasonic diagnostic apparatus 1 of the embodiment has an ultrasonic probe 31, an operation console 32, and a display unit 41. The ultrasonic diagnostic apparatus 1 of the embodiment generates a plurality of frames in chronological order of a slice image of a subject on the basis of an echo signal obtained by performing a scan of transmitting an ultrasonic wave to the subject and receiving the ultrasonic wave reflected from the subject to which the ultrasonic wave is transmitted, and sequentially displays the frames. The components will be described one by one.

The ultrasonic probe 31 includes a plurality of ultrasonic transducers (not shown) which are, for example, arranged uniformly in a matrix. The ultrasonic transducers in the ultrasonic probe 31 are constructed by including, for example, a piezoelectric material such as lead zirconium titanate (PZT) ceramics. The ultrasonic transducers convert an electric signal to a sound wave, transmit the sound wave, and convert a received sound wave to an electric signal. The ultrasonic probe 31 is used by making its surface in which the ultrasonic transducers are formed in contact with the surface of a subject. As will be specifically described later, the ultrasonic probe 31 performs a scan by transmitting an ultrasonic wave from the ultrasonic transducers into the subject in accordance with a drive signal from a transmitting/receiving unit 32 based on a control signal output from a control unit 324 in the operation console 32, and receiving the ultrasonic wave reflected from the subject to which the ultrasonic wave has been transmitted by the ultrasonic transducers, thereby obtaining an echo signal. The ultrasonic probe 31 outputs the echo signal to the transmitting/receiving unit 321.

The operation console 32 has, as shown in FIG. 1, the transmitting/receiving unit 321, an image generating unit 322, a storing unit 323, the control unit 324, an operation unit 325, a start frame selecting unit 326, and an injection time counting unit 327. Each of the components of the operation console 32 includes a data processor, and the operation console 32 executes processes on various data.

The transmitting/receiving unit 321 includes a transmission/reception circuit for making the ultrasonic probe 31 transmit/receive an ultrasonic wave, makes the ultrasonic transducers in the ultrasonic probe 31 transmit an ultrasonic wave to the subject, and makes the ultrasonic transducers receive the ultrasonic wave reflected from the subject, thereby obtaining an echo signal. For example, the transmitting/receiving unit 321 performs a scan on the subject in an electronic convex scan method to obtain an echo signal and outputs the obtained echo signal to the image generating unit 322. Concretely, the transmitting/receiving unit 321 obtains an echo signal by driving while switching the positions of the plurality of ultrasonic transducers of the ultrasonic probe 31 so as to scan the subject while moving an ultrasonic beam, performs processes such as amplification, delay, and addition on the echo signal, and outputs the processed echo signal to the image generating unit 322.

The image generating unit 322 generates a slice image of a slice face of the subject. Concretely, the image generating unit 322 includes a logarithmic amplifier and an envelope detector, logarithmic-amplifies the echo signal output from the transmitting/receiving unit 321 and, after that, detects an envelope. After that, the image generating unit 322 performs a predetermined data process on the data, calculates the intensity of an echo from each of reflection points on a sound ray, converts the intensity to a brightness value, and generates a slice image corresponding to the B mode. In the embodiment, on the basis of a control signal from the control unit 324, the image generating unit 322 performs a data process on an echo signal from the transmitting/receiving unit 321, and generates a plurality of frames in chronological order from the slice image, thereby generating a moving picture. The image generating unit 322 is connected to the storing unit 323 and outputs the moving picture generated as described above on the frame unit basis to the storing unit 323.

The storing unit 323 is constructed so as to include, for example, a cine memory and an HDD and stores data of an image generated by the image generating unit 322. The storing unit 323 is connected to the image generating unit 322. On the basis of an instruction from the control unit 324, the storing unit 323 temporarily stores the slice image of the plurality of frames generated as a moving picture by the image generating unit 322 into the cine memory and, after that, outputs and stores it to the HDD. For example, the storing unit 323 stores images of frames corresponding to moving pictures of the amount of two minutes in the cine memory, and outputs and stores the images of frames of the moving pictures of the amount of two minutes into the HDD. The cine memory in the storing unit 323 is connected to the display unit 41, and the data of the slice image of each frame of the moving pictures stored in the cine memory is output to the display unit 41. The HDD in the storing unit 323 is similarly connected to the display unit 41. On the basis of an instruction entered by the operator to the operation unit 325, the data of the slice image of each of frames of the moving pictures stored in the HDD is output to the display unit 41.

The control unit 324 includes, for example, a computer and a program for making the computer execute a predetermined data process, and is connected to the components. In the embodiment, based on an operation signal from the operation unit 325, the control unit 324 supplies control signals to the components to control their operations.

The operation unit 325 includes input devices such as, for example, a keyboard, a touch panel, a track ball, a foot switch, and a sound input device. Operation information is input by the operator to the operation unit 325. Based on the operation information, the operation unit 325 outputs an operation signal to the control unit 324.

The start frame selecting unit 326 includes a computer and a program for making the computer execute a predetermined data process, and selects a start frame used when the display unit 41 starts chronological-order display of the slice images of the plurality of frames generated in chronological order by the image generating unit 322. In the embodiment, the start frame selecting unit 326 selects the start frame so as to correspond to the time point when the injection time counting unit 327 starts counting time of injecting a contrast medium into the subject. In this case, a frame generated by the image generating unit 322 when the injection time counting unit 327 starts counting time of injecting a contrast medium into the subject is selected as a start frame.

The injection time counting unit 327 includes a timer and counts time in which a contrast medium is injected to a fluid flowing in the subject. In the embodiment, time is counted by using, as a trigger, the time point when injection of the contrast medium into blood flowing in the blood vessels of the subject starts.

The display unit 41 includes, for example, an LCD device (not shown) having a plane display screen and a DSC (Digital Scan Converter), and displays an image generated by the image generating unit 322 and stored in the storing unit 323. The display unit 41 sequentially displays the slice images of the plurality of frames stored in the storing unit 323 in accordance with the chronological order in which the image generating unit 322 has generated the plurality of frames. Concretely, the display unit 41 is connected to the storing unit 323. Based on an instruction from the control unit 324, the display unit 41 converts data of the slice image of each of the frames stored in the cine memory in the storing unit 323 to a display signal by the DSC, and displays the resultant as a slice image on the display screen of the LCD device. The display unit 41 is also connected to the HDD in the storing unit 323. Based on an instruction which is input to the operation unit 325 by the operator, the display unit 41 receives image data of the moving picture stored in the HDD and displays the picture on the screen. In the embodiment, the display unit 41 sequentially displays slice images of frames generated in chronological order by the image generating unit 322 after the slice image of the start frame selected by the start frame selecting unit 326.

Operations

Operations performed at the time of imaging the subject by using the ultrasonic diagnostic apparatus 1 of the embodiment of the invention will be described below.

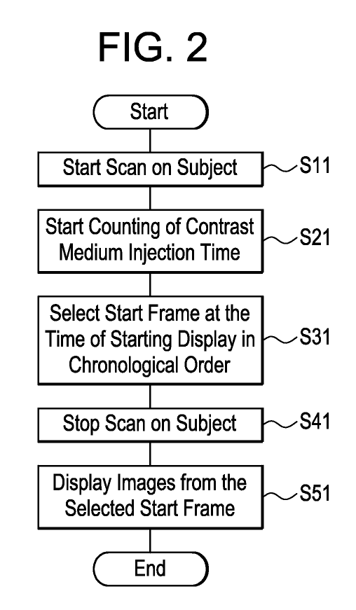

FIG. 2 is a flowchart showing operations performed at the time of imaging the subject in the first embodiment of the invention.

First, as shown in FIG. 2, a scan on the subject is started (S11).

The operator makes the surface in which the ultrasonic transducers are arranged in the ultrasonic probe 31 come into contact with an image acquisition region of the subject, and inputs an instruction of starting a scan on the subject to the operation unit 325. The control unit 324 controls the components so as to start a scan on the subject.

Concretely, the transmitting/receiving unit 321 transmits ultrasonic waves from the ultrasonic transducers of the ultrasonic probe 31 to the subject, and receives the ultrasonic waves reflected from the subject by the ultrasonic transducers, thereby obtaining echo signals. After that, the image generating unit 322 processes the echo signals from the transmitting/receiving unit 321 to generate slice images in a plurality of frames in chronological order. The slice images of the plurality of frames generated as moving pictures by the image generating unit 322 are stored in the cine memory in the storing unit 323. The display unit 41 displays the slice images of the frames of the motion pictures stored in the cine memory so as to be real-time to the scan.

Next, as shown in FIG. 2, counting of the contrast medium injection time is started (S21).

In the embodiment, a contrast medium is injected to the blood flowing in the blood vessels of the subject. By using the time point when the injection of the contrast medium is started as a trigger, the injection time counting unit 327 counts the time in which the contrast medium is injected.

Next, as shown in FIG. 2, a start frame at the time of starting display in chronological order is selected (S31).

The start frame selecting unit 326 selects the start frame at the time of starting chronological-order display of the slice images of the plurality of frames generated in chronological order by the image generating unit 322 on the display unit 41.

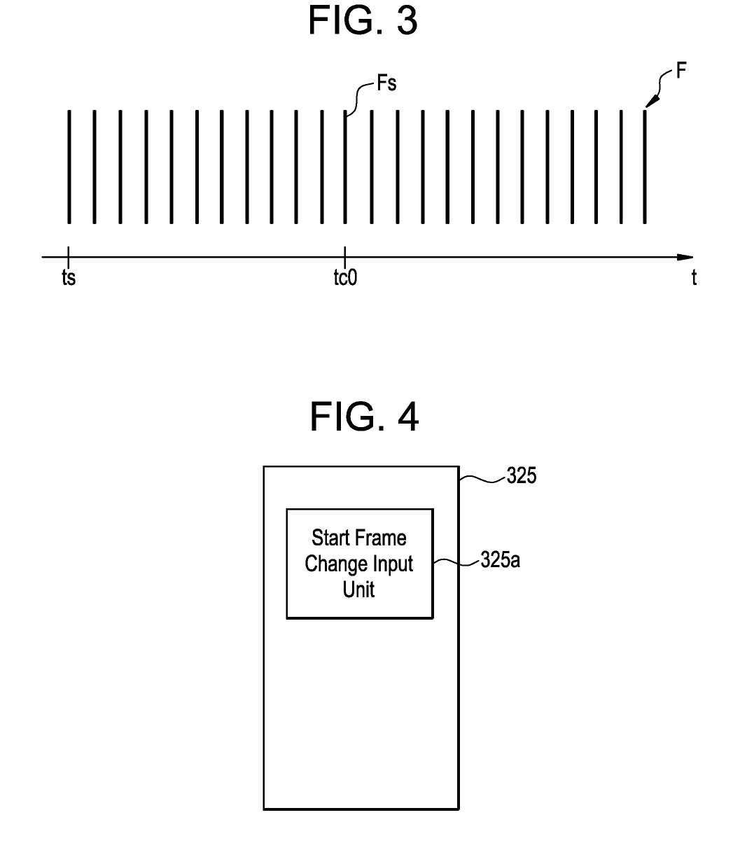

FIG. 3 is a diagram showing a state where the start frame selecting unit 326 selects a start frame at the time of starting display in chronological order in the first embodiment of the invention. In FIG. 3, the horizontal axis denotes time base “t”.

In the embodiment, as shown in FIG. 3, the start frame selecting unit 326 selects, as a start frame Fs, a frame generated by the image generating unit 322 at time point tc0 when the injection time counting unit 327 starts counting the time of injecting the contrast medium into the subject, from slice images in a plurality of frames F generated in chronological order by the image generating unit 322 after the scan start time ts. While the slice images of frames are stored in the cine memory, the start frame selecting unit 326 selects the start frame.

Next, as shown in FIG. 2, the scan on the subject is stopped (S41).

The operator inputs an instruction of finishing the scan on the subject to the operation unit 325, and the control unit 324 controls the components so as to stop the scan.

Subsequently, as shown in FIG. 2, the images are displayed from the selected start frame (S51).

The display unit 41 sequentially displays the slice images of the plurality of frames stored in the storing unit 323 onto the display screen in accordance with the chronological order in which the image generating unit 322 has generated the plurality of frames.

In the embodiment, the display unit 41 sequentially displays the slice images of frames generated in chronological order by the image generating unit 322, which are the slice image of the start frame Fs selected by the start frame selecting unit 326 and the subsequent slice images as shown in FIG. 3.

As described above, in the embodiment, the start frame selecting unit 326 selects the start frame Fs corresponding to the time the display unit 41 starts displaying the slice images in chronological order, from the slice images of the plurality of frames F generated in chronological order by the image generating unit 322. The start frame selecting unit 326 selects the start frame Fs in accordance with the time point tc0 when the injection time counting unit 327 starts counting the time of injecting the contrast medium into the subject. Concretely, the frame F generated by the image generating unit 322 when the injection time counting unit 327 starts the counting is selected as the start frame Fs. The display unit 41 sequentially displays the slice image of the start frame Fs selected by the start frame selecting unit 326 and the subsequent slice images of the frames generated in chronological order by the image generating unit 322. Thus, in the embodiment, since complicated operations are unnecessary at the time of starting sequential reproduction of slice images of the frames in a necessary range, the operation efficiency improves, and high diagnosis efficiency can be realized.

### Second Embodiment

A second embodiment of the present invention will be described below.

FIG. 4 is a block diagram showing the operation unit 325 in the second embodiment of the invention.

As shown in FIG. 4, the operation unit 325 of the second embodiment has a start frame change input unit 325a. The second embodiment is similar to the first embodiment except for this point. Consequently, description of the same parts will not be repeated.

The start frame change input unit 325a is provided for the operation unit 325. An instruction of changing the position of the start frame selected by the start frame selecting unit 326 is entered by the operator.

When the instruction of changing the position of the start frame is input to the start frame change input unit 325a, the start frame selecting unit 326 selects a start frame so as to respond to the instruction of changing the position of the start frame that is input to the start frame change input unit 325a.

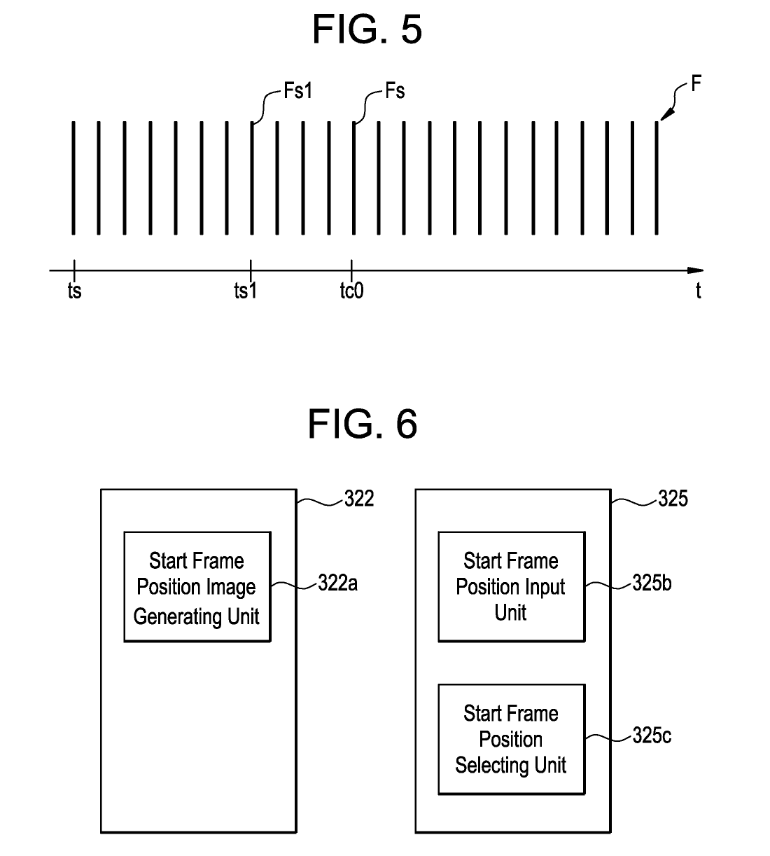

FIG. 5 is a diagram showing a state where the start frame selecting unit 326 selects a start frame at the time of starting display in chronological order in the second embodiment of the invention. In FIG. 5, the horizontal axis indicates the time base “t”.

In the second embodiment, as shown in FIG. 5, the start frame selecting unit 326 changes the start frame Fs selected so as to correspond to the count start time tc0 by the start frame selecting unit 326 to a start frame at the time point ts1 corresponding to the instruction of changing the position of the start frame, which is input to the start frame change input unit 325a and selects it in slice images in a plurality of frames F generated in chronological order by the image generating unit 322 after the scan start time ts. The display unit 41 sequentially displays the slice image of the start frame Fs selected by the start frame selecting unit 326 and the subsequent slice images of the frames generated in chronological order by the image generating unit 322.

As described above, in the second embodiment, the start frame selecting unit 326 newly selects, as the start frame, the frame generated at the time point corresponding to the instruction of changing the position of the start frame, which is input to the start frame change input unit 325a. Consequently, in the second embodiment, at the time of starting sequential reproduction of slice images of frames F in a necessary range, complicated operations are unnecessary. Thus, operation efficiency improves and high diagnosis efficiency can be realized.

### Third Embodiment

A third embodiment of the present invention will be described hereinbelow.

FIG. 6 is a block diagram showing the image generating unit 322 and the operation unit 325 in the third embodiment of the invention.

As shown in FIG. 6, the image generating unit 322 of the third embodiment has the start frame position image generating unit 322a and, on the other hand, the operation unit 325 of the third embodiment has a start frame position input unit 325b and a start frame position selecting unit 325c. The third embodiment is similar to the second embodiment except for this point. Consequently, description of the same parts will not be repeated.

The start frame position image generating unit 322a includes a computer and a program for making the computer execute a predetermined data process, and generates a start frame position image indicative of the position of the start frame which is input by the start frame position input unit 325b in slice images of the plurality of frames generated in chronological order by the image generating unit 322.

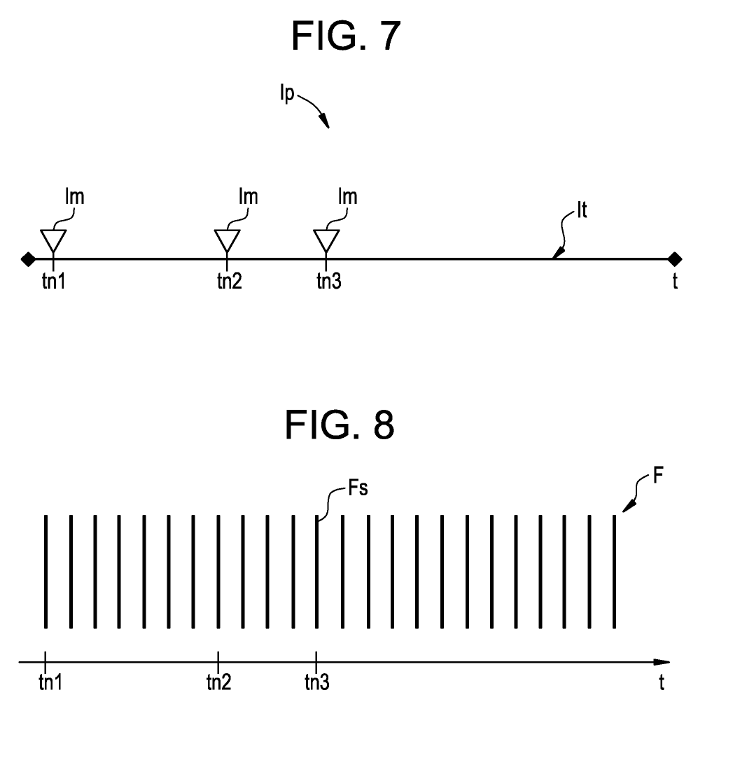

FIG. 7 is a diagram showing start frame position images generated by the start frame position image generating unit 322a in the third embodiment of the invention.

As shown in FIG. 7, the frame position image generating unit 322a generates a start frame position image Ip by superimposing a start mark image Im indicating, by a mark, the position of a start frame which is input by the start frame position input unit 325b onto a time bar image It indicative of the time base along which the image generating unit 322 has generated slice images in a plurality of frames. For example, in the case where three time points tn1, tn2, and tn3 are input as positions of start frames, a start frame position image Ip is generated by superimposing the mark images Im on the time bar image It at the time points. The generated start frame position image Ip is output to the display unit 41. The start frame position image Ip is displayed by the display unit 41.

The start frame position input unit 325b is provided for the operation unit 325 and, based on an instruction from the operator, inputs the position of a start frame from which the display unit 41 starts displaying in the chronological order in which the image generating unit 322 has generated slice images in a plurality of frames.

The start frame position selecting unit 325c is provided for the operation unit 325. In the case where a plurality of positions of start frames are input by the start frame position input unit 325c, the start frame position selecting unit 325c selects one of the positions of the plurality of start frames on the basis of the instruction from the operator.

In the third embodiment, in the case where the position of the start frame is input by the start frame position input unit 325b, the start frame selecting unit 326 selects a start frame so as to be adapted to the position of the start frame which is input by the start frame position input unit 325b. In this case, the start frame selecting unit 326 selects the start frame so as to be adapted to the position of one start frame selected by the start frame position selecting unit 325c.

FIG. 8 is a diagram showing a state where the start frame selecting unit 326 selects a start frame at the time of starting display in chronological order in the third embodiment of the invention. In FIG. 8, the horizontal axis denotes time base “t”.

In the third embodiment, as shown in FIG. 8, by using the start frame position input unit 325b, the operator inputs a plurality of positions of start frames in chronological order in which the image generating unit 322 has generated slice images of the plurality of frames in the slice images of the plurality of frames F generated in chronological order by the image generating unit 322 since the scan start time point ts. For example, as shown in FIG. 8, first, second, and third time points tn1, tn2, and tn3 are input as positions of start frames. By using the start frame position selecting unit 325c, the operator selects one of the positions of the plurality of start frames which are input by the start frame position input unit 325c. For example, the operator selects the third time point tn3. After that, the start frame selecting unit 326 selects, as the start frame Fs, a frame corresponding to the third time point tn3 as the position of the one start frame selected by the start frame position selecting unit 325c. The display unit 41 sequentially displays the slice image of the start frame Fs selected by the start frame selecting unit 326 and the subsequent slice images of the frames generated in chronological order by the image generating unit 322.

As described above, in the third embodiment, the start frame selecting unit 326 selects a start frame so as to correspond to the position of the start frame which is input by the start frame position input unit 325b. In this case, the start frame is selected so as to correspond to the position of the one start frame selected by the start frame position selecting unit 325c. In the third embodiment, at the time of starting sequential reproduction of the slice images of the frames F in a necessary range, complicated operations are unnecessary. Thus, operation efficiency improves and high diagnostic efficiency can be realized.

### Fourth Embodiment

A fourth embodiment of the present invention will be described below.

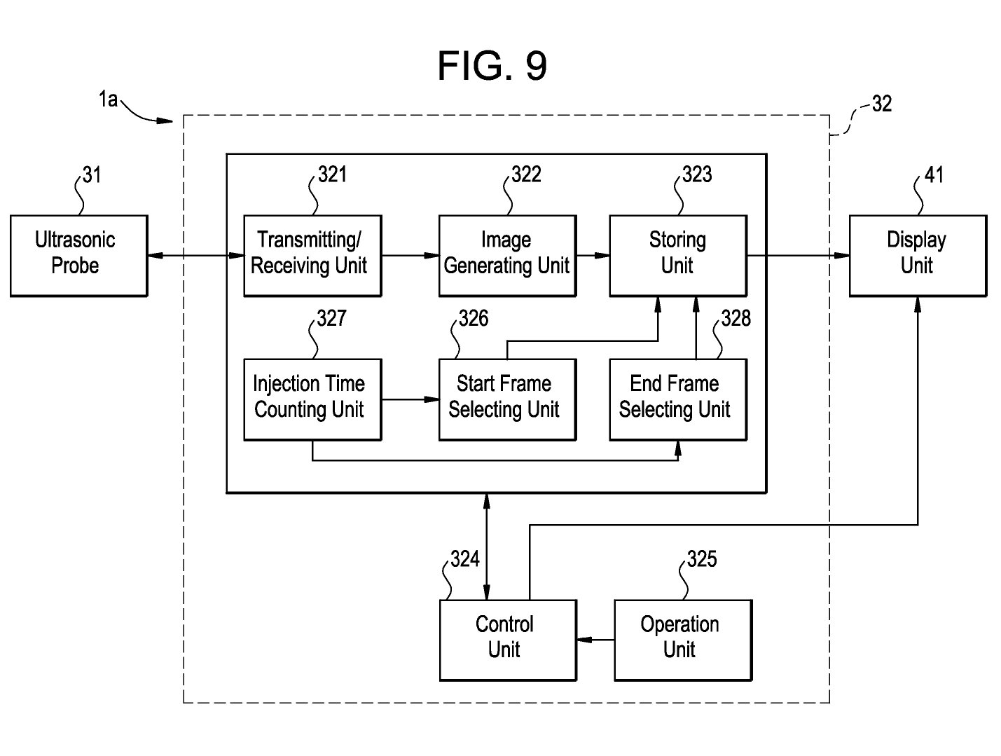

FIG. 9 is a block diagram showing the configuration of an ultrasonic diagnostic apparatus 1a in the fourth embodiment of the invention.

As shown in FIG. 9, in the ultrasonic diagnostic apparatus 1a of the fourth embodiment, the operation console 32 includes an end frame selecting unit 328. The fourth embodiment is similar to the third embodiment except for this point. Consequently, description of the same parts will not be repeated.

The end frame selecting unit 328 includes a computer and a program for making the computer execute a predetermined data process, and selects an end frame at the time of finishing display of slice images in chronological order started by the display unit 41 in slice images in a plurality of frames generated in chronological order by the image generating unit 322. In the case where the injection time counting unit 327 finishes counting of time in which a contrast medium is injected into the subject, the end frame selecting unit 328 selects the end frame so as to correspond to the time point when the injection time counting unit 327 finishes the counting of time in which the contrast medium is injected into the subject. In the fourth embodiment, the end frame selecting unit 328 selects, as an end frame, a frame generated by the image generating unit 322 when the injection time counting unit 328 finishes the counting of the time in which the contrast medium is injected into the subject.

The display unit 41 sequentially displays slice images of frames generated in chronological order by the image generating unit 322 up to the slice image of the end frame selected by the end frame selecting unit 328.

Operations performed at the time of imaging the subject by using the ultrasonic diagnostic apparatus 1 a of the fourth embodiment of the invention will be described below.

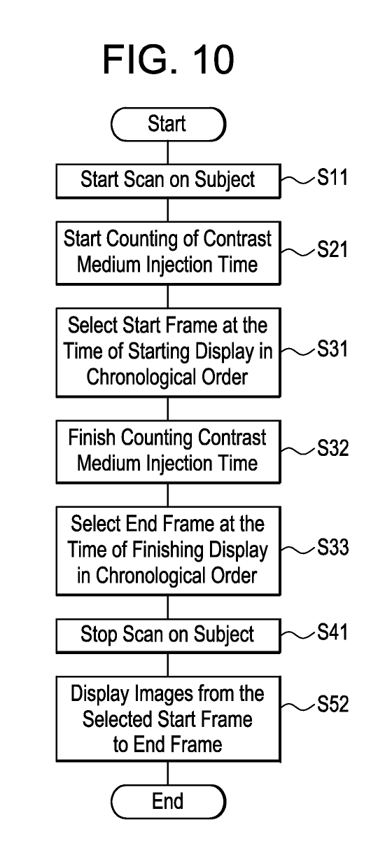

FIG. 10 is a flowchart showing operations performed at the time of imaging the subject in the fourth embodiment of the invention.

First, as shown in FIG. 4, in a manner similar to the first embodiment, a scan on the subject is started (S11) and, after that, counting of time in which a contrast medium is injected is started (S21). Then, a start frame at the time of starting display in chronological order is selected (S31).

Next, as shown in FIG. 10, the counting of the contrast medium injection time is finished (S32).

The injection time counting unit 327 finishes the counting of the time in which the contrast medium is injected by using, as a trigger, the time point when the injection of the contrast medium to the blood flowing in the blood vessels of the subject is finished.

Next, as shown in FIG. 10, the end frame at the time of finishing the display in the chronological order is selected (S33).

In the embodiment, the end frame selecting unit 328 selects, as the end frame, a frame generated by the image generating unit 322 at the time when the injection time counting unit 328 finishes the counting of the time in which the contrast medium is injected into the subject.

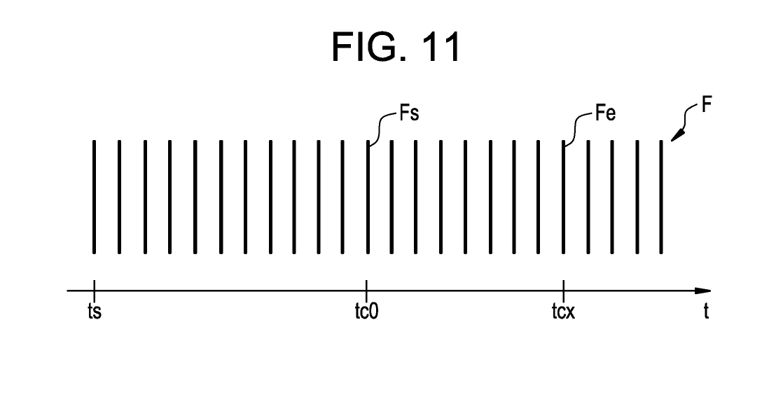

FIG. 11 is a diagram showing a state where the end frame selecting unit 328 selects a start frame at the time of finishing display in chronological order in the fourth embodiment of the invention. In FIG. 11, the horizontal axis denotes time base “t”.

In the fourth embodiment, as shown in FIG. 11, the end frame selecting unit 326 selects, as an end frame Fe, a frame generated by the image generating unit 322 at time point tcx when the injection time counting unit 327 finishes counting the time of injecting the contrast medium into the subject, in slice images in a plurality of frames F generated in chronological order by the image generating unit 322 after the scan start time ts.

Next, as shown in FIG. 10, the scan on the subject is stopped (S41).

In a manner similar to the first embodiment, the operator inputs an instruction of finishing the scan on the subject to the operation unit 325, and the control unit 324 controls the components so as to stop the scan.

Subsequently, as shown in FIG. 10, the images are displayed from the selected start frame to the end frame (S52).

The display unit 41 sequentially displays the slice images of the plurality of frames stored in the storing unit 323 onto the display screen in accordance with the chronological order in which the image generating unit 322 has generated the plurality of frames.

In the fourth embodiment, the display unit 41 sequentially displays the slice images of frames generated in chronological order by the image generating unit 322, starting from the slice image of the start frame Fs selected by the start frame selecting unit 326 and ended by the slice image of the end frame Fe selected by the end frame selecting unit 328 as shown in FIG. 11.

As described above, in the fourth embodiment, the display unit 41 sequentially displays the slice images of the frames generated in chronological order by the image generating unit 322 up to the slice image of the end frame selected by the end frame selecting unit 328. Thus, in the fourth embodiment, complicated operations are unnecessary at the time of starting sequential reproduction of slice images of the frames F in a necessary range, so that the operation efficiency improves, and high diagnosis efficiency can be realized.

### Fifth Embodiment

A fifth embodiment of the present invention will be described below.

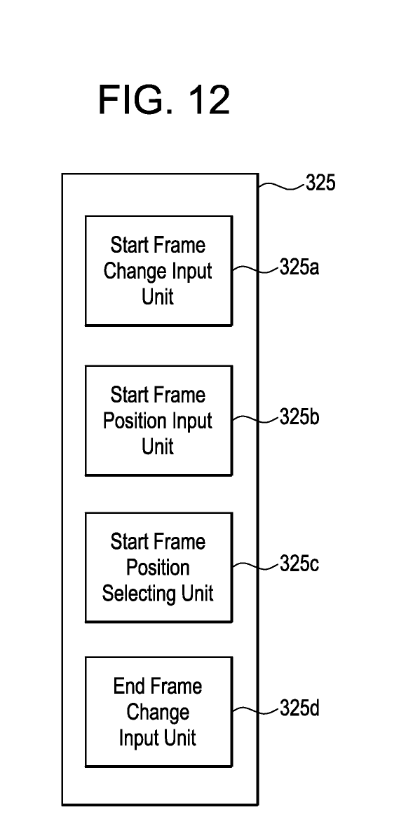

FIG. 12 is a block diagram showing the operation unit 325 in the fifth embodiment of the invention.

As shown in FIG. 12, the operation unit 325 of the fifth embodiment has an end frame change input unit 325d. The fifth embodiment is similar to the fourth embodiment except for this point. Consequently, description of the same parts will not be repeated.

The end frame change input unit 325d is provided for the operation unit 325. An instruction of changing the position of the end frame selected by the end frame selecting unit 328 is entered by the operator.

When the instruction of changing the position of the end frame is input to the end frame change input unit 325c, the end frame selecting unit 328 selects an end frame so as to respond to the instruction of changing the position of the end frame that is input to the end frame change input unit 325c.

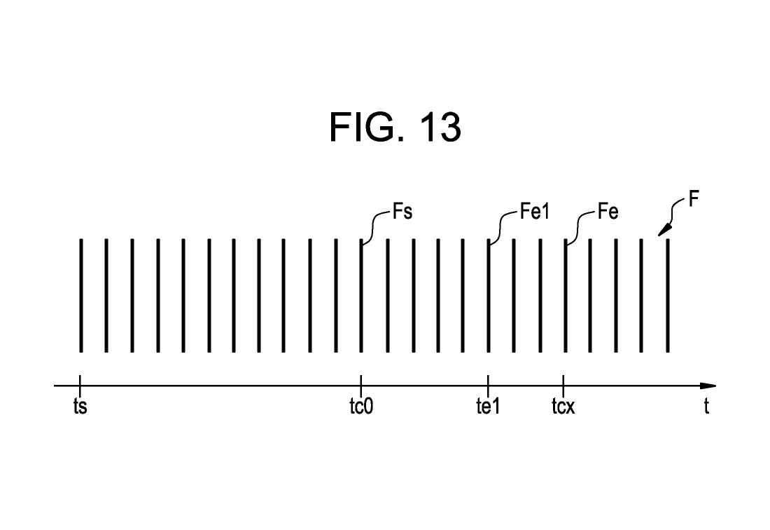

FIG. 13 is a diagram showing a state where the end frame selecting unit 328 selects an end frame at the time of finishing display in chronological order in the fifth embodiment of the invention. In FIG. 13, the horizontal axis indicates the time base “t”.

In the fifth embodiment, as shown in FIG. 13, the end frame selecting unit 328 changes the end frame Fe selected so as to correspond to the count end time tcx by the end frame selecting unit 328 to an end frame at the time point te1 corresponding to the instruction of changing the position of the end frame, which is input to the end frame change input unit 325d and selects it in slice images in a plurality of frames F generated in chronological order by the image generating unit 322 since the scan start time ts. The display unit 41 sequentially displays the slice images of the frames generated in chronological order by the image generating unit 322 up to the slice image of the end frame Fe1 selected by the end frame selecting unit 328.

As described above, in the fifth embodiment, the end frame selecting unit 328 newly selects, as the end frame, the frame generated by the image generating unit 322 at the time point corresponding to the instruction of changing the position of the end frame, which is input to the end frame change input unit 325d. Consequently, in the fifth embodiment, at the time of starting sequential reproduction of slice images of frames F in a necessary range, complicated operations are unnecessary. Thus, operation efficiency improves and high diagnosis efficiency can be realized.

### Sixth Embodiment

A sixth embodiment of the present invention will be described hereinbelow.

FIG. 14 is a block diagram showing the image generating unit 322 and the operation unit 325 in the sixth embodiment of the invention.

As shown in FIG. 14, the image generating unit 322 of the sixth embodiment has an end frame position image generating unit 322b and, on the other hand, the operation unit 325 of the sixth embodiment has an end frame position input unit 325e and an end frame position selecting unit 325f. The sixth embodiment is similar to the fifth embodiment except for this point. Consequently, description of the same parts will not be repeated.

The end frame position image generating unit 322b includes a computer and a program for making the computer execute a predetermined data process, and generates an end frame position image indicative of the position of the end frame which is input by the end frame position input unit 325e in slice images of the plurality of frames generated in chronological order by the image generating unit 322.

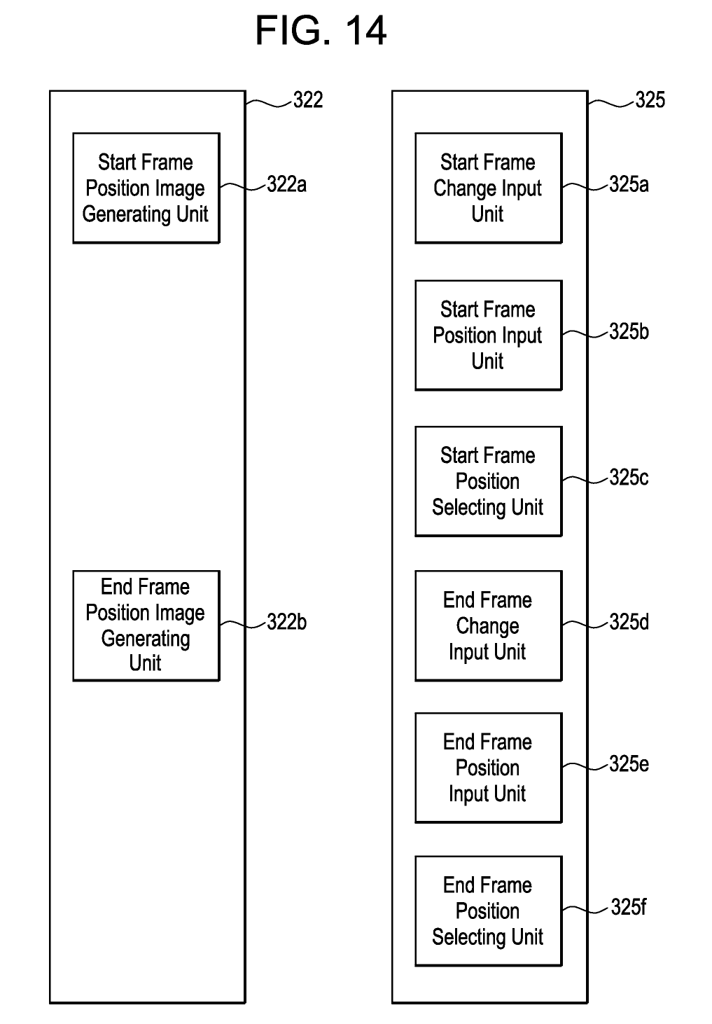

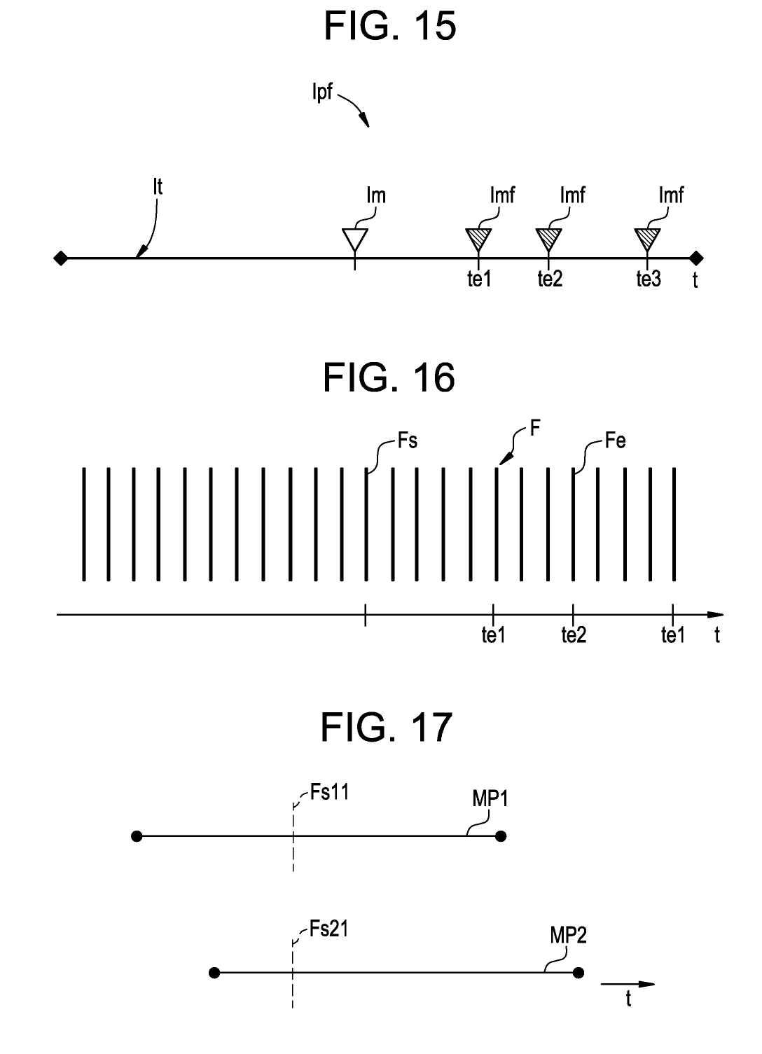

FIG. 15 is a diagram showing end frame position images generated by the end frame position image generating unit 322b in the sixth embodiment of the invention.

As shown in FIG. 15, the end frame position image generating unit 322b generates an end frame position image Ipf by superimposing an end mark image Imf indicating, by a mark, the position of an end frame which is input by the end frame position input unit 325e onto a time bar image It indicative of the time base along which the image generating unit 322 has generated slice images in a plurality of frames. For example, in the case where three time points te1, te2, and te3 are input as positions of end frames, an end frame position image Ipf is generated by superimposing the mark images Imf on the time bar image It at the time points. The generated end frame position image Ipf is output to the display unit 41. The end frame position image Ipf is displayed by the display unit 41.

The end frame position input unit 325e is provided for the operation unit 325 and, based on an instruction from the operator, inputs the position of an end frame for finishing the display in the chronological order in which the image generating unit 322 has generated slice images in a plurality of frames.

The end frame position selecting unit 325f is provided for the operation unit 325. In the case where a plurality of positions of end frames are input by the end frame position input unit 325f, the end frame position selecting unit 325f selects one of the positions of the plurality of end frames on the basis of the instruction from the operator.

In the sixth embodiment, in the case where the position of the end frame is input by the end frame position input unit 325e, the end frame selecting unit 328 selects an end frame so as to correspond to the position of the end frame which is input by the end frame position input unit 325e. In this case, the end frame selecting unit 328 selects the end frame so as to correspond to the position of one end frame selected by the end frame position selecting unit 325f.

FIG. 16 is a diagram showing a state where the end frame selecting unit 328 selects an end frame at the time of finishing display in chronological order in the sixth embodiment of the invention. In FIG. 16, the horizontal axis denotes time base “t”.

In the sixth embodiment, as shown in FIG. 16, by using the end frame position input unit 325e, the operator inputs a plurality of positions of end frames in chronological order in which the image generating unit 322 has generated slice images of the plurality of frames in the slice images of the plurality of frames F generated in chronological order by the image generating unit 322 since the scan start time point ts. For example, as shown in FIG. 16, first, second, and third time points te1, te2, and te3 are input as positions of start frames. By using the end frame position selecting unit 325f, the operator selects one of the positions of the plurality of end frames which are input by the end frame position input unit 325e. For example, the operator selects the second time point te2. After that, the end frame selecting unit 328 selects, as the end frame Fe, a frame corresponding to the third time point te3 as the position of the one end frame selected by the end frame position selecting unit 325f. The display unit 41 sequentially displays the slice images of the frames generated in chronological order by the image generating unit 322 up to the slice image of the end frame Fe selected by the end frame selecting unit 328.

As described above, in the sixth embodiment, the end frame selecting unit 328 selects an end frame so as to correspond to the position of the end frame which is input by the end frame position input unit 325e. In this case, the end frame is selected so as to correspond to the position of the one end frame selected by the end frame position selecting unit 325f. In the sixth embodiment, the slice images of the frames F in a necessary range can be sequentially reproduced, so that complicated operations are unnecessary. Thus, operation efficiency improves and high diagnostic efficiency can be realized.

### Seventh Embodiment

A seventh embodiment of the invention will be described below.

The seventh embodiment is different from the first embodiment with respect to the operations of the ultrasonic diagnostic apparatus 1. The seventh embodiment is similar to the first embodiment except for this point. Description of the same parts will not be repeated.

In the seventh embodiment, based on echo signals obtained by scanning the subject, the image generating unit 322 generates a plurality of frames in chronological order of slice images of the subject, thereby generating a plurality of moving pictures. For example, the image generating unit 322 generates a first moving picture MP1 and a second moving picture MP2. The storing unit 323 stores the moving pictures MP1 and MP2.

After that, the start frame selecting unit 326 selects, as a start frame, a first start frame Fs11 when the display unit 41 starts displaying the slice images in chronological order, in the first moving picture MP1 generated by the image generating unit 322. The start frame selecting unit 326 also selects a second start frame Fs21 when the display unit 41 starts displaying the slice images in chronological order, in the second moving picture MP2 generated by the image generating unit 322. Alternatively, a frame from which a slice image is not generated may be selected as a virtual start frame.

The display unit 41 displays the first moving picture MP1 and the second moving picture MP2 side by side on the display screen.

FIG. 17 is a diagram showing a state where the display unit 41 displays the first moving picture MP1 and the second moving picture MP2 side by side on the display screen in the seventh embodiment of the invention. In FIG. 17, the image acquisition time in which the first and second moving pictures MP1 and MP2 are acquired is shown by the horizontal axis, and the start frames selected in the first and second moving pictures by the start frame selecting unit 326 are shown by dot lines.

As shown in FIG. 17, in the seventh embodiment, the display unit 41 sequentially displays the slice image of the first frame Fs11 selected by the start frame selecting unit 326 in the first moving picture MP1 and the subsequent slice images of frames generated in chronological order by the image generating unit 322, and the slice image of the second start frame Fs21 selected by the start frame selecting unit 326 in the second moving picture MP2 and the subsequent slice images of frames generated by the image generating unit 322 so that the first and second start frames Fs11 and Fs22 are the same on the time base.

As described above, in the seventh embodiment, even in the case where the positions of frames in a display range are various in time series among a plurality of moving pictures, the images of the frames in the display range can be easily displayed synchronously. Consequently, in the seventh embodiment, complicated operations are unnecessary. Thus, operation efficiency improves and high diagnosis efficiency can be realized.

The ultrasonic probe 31 in the embodiments corresponds to the scanning unit of the present invention. The display unit 41 in the embodiments corresponds to the display unit of the present invention. The image generating unit 322 in the embodiments corresponds to the image generating unit of the present invention. The storing unit 323 in the embodiments corresponds to the storing unit of the invention. The start frame selecting unit 326 in the embodiments corresponds to the start frame selecting unit of the invention. The injection time counting unit 327 in the embodiments corresponds to the injection time counting unit of the invention. The end frame selecting unit 328 in the embodiments corresponds to the end frame selecting unit of the invention. The start frame position image generating unit 322a of the embodiments corresponds to the start frame position image generating unit of the invention. The end frame position image generating unit 322b in the embodiments corresponds to the end frame position image generating unit of the invention. The start frame change input unit 325a in the embodiments corresponds to the start frame change input unit of the invention. The start frame position input unit 325b in the embodiments corresponds to the start frame position input unit of the invention. The start frame position selecting unit 325c in the embodiments corresponds to the start frame position selecting unit of the invention. The end frame change input unit 325d in the embodiments corresponds to the end frame change input unit of the invention. The end frame position input unit 325e in the embodiments corresponds to the end frame position input unit of the invention. The end frame position selecting unit 325f in the embodiments corresponds to the end frame position selecting unit of the invention.

The present invention is not limited to the foregoing embodiments but various modifications can be employed.

For example, the components of the embodiments may be combined.

For example, the invention can be also applied to the case where the position of a start frame in chronological order in which the image generating unit 322 generates slice images in a plurality of frames is input by the operator using the start frame position input unit 325b before a scan by the ultrasonic probe 31 is performed. In this case, the start frame selecting unit 326 selects a start frame so as to correspond to the position of the start frame which has been input in advance by the start frame position input unit 325b. For example, a start frame corresponding to a time point before or after lapse of predetermined time from the time point when the injection time counting unit 327 starts counting the time of injection of the contrast medium into the subject. Concretely, a frame corresponding to a time point five seconds before the time point when the injection time counting unit 327 starts counting the time in which the contrast medium is injected into the subject is selected as a start frame. For example, a frame corresponding to a time point after lapse of 30 seconds from the time point when the injection time counting unit 327 starts counting the time in which the contrast medium is injected into the subject is selected as a start frame.

Similarly, the invention can be also applied to the case where the position of an end frame in chronological order in which the image generating unit 322 generates slice images in a plurality of frames is input by the operator using the end frame position input unit 325c before a scan by the ultrasonic probe 31 is performed. In this case, the end frame selecting unit 328 selects an end frame so as to correspond to the position of the end frame which has been input in advance by the end frame position input unit 325c. For example, an end frame corresponding to a time point before or after lapse of predetermined time from the time point when the injection time counting unit 327 finishes counting the time of injection of the contrast medium into the subject.

Many widely different embodiments of the invention may be configured without departing from the spirit and the scope of the present invention. It should be understood that the present invention is not limited to the specific embodiments described in the specification, except as defined in the appended claims.

1. An ultrasonic diagnostic apparatus comprising:
a scanning device for obtaining echo signals by performing a scan of transmitting ultrasonic waves to a subject into which a contrast medium is injected and receiving the ultrasonic waves reflected from the subject to which the ultrasonic waves were transmitted;
an image generating device for generating slice images of the subject in a plurality of frames in chronological order on the basis of the echo signals obtained by the scanning device;
a storing device for storing the slice images in the plurality of frames generated by the image generating device; and
a display device for sequentially displaying the slice images in the plurality of frames stored in the storing device so as to correspond to the chronological order in which the image generating device has generated the plurality of frames,
wherein the apparatus comprises:
a start frame selecting device for selecting a start frame at the time the display device starts displaying the slice images in the chronological order from the slice images in the plurality of frames generated in the chronological order by the image generating device; and
an injection time counting device for counting time in which the contrast medium is injected into the subject,
the start frame selecting device selects, as the start frame, a frame generated by the image generating device when the injection time counting device starts counting the time in which the contrast medium is injected into the subject, and
the display device sequentially displays the slice image of the start frame selected by the start frame selecting device and the subsequent slice images in the frames generated in the chronological order by the image generating device.

2. The ultrasonic diagnostic apparatus according to claim 1, further comprising a start frame change input device to which an instruction of changing the position of the start frame selected by the start frame selecting device is input by the operator,
wherein in the case where an instruction of changing the position of the start frame is input to the start frame change input device, the start frame selecting device selects a start frame so as to correspond to the instruction of changing the position of the start frame input to the start frame change input device.

3. The ultrasonic diagnostic apparatus according to claim 1, further comprising a start frame position input device for inputting the position of the start frame in the slice images in the plurality of frames generated in chronological order by the image generating device on the basis of an instruction from the operator,
wherein in the case where the position of the start frame is input by the start frame position input device, the start frame selecting device selects the start frame so as to correspond to the position of the start frame which is input by the start frame position input device.

4. The ultrasonic diagnostic apparatus according to claim 3, wherein the position of the start frame in the chronological order in which the image generating device generates the slice images of the plurality of frames is input to the start frame position input device by the operator before a scan is performed by the scanning device.

5. The ultrasonic diagnostic apparatus according to claim 3, further comprising a start frame position selecting device, when a plurality of positions of the start frames are input by the start frame position input device, for selecting position of one of the plurality of start frames on the basis of an instruction from the operator,
wherein when the position of one of the start frames is selected by the start frame position selecting device, the start frame selecting device selects the start frame so as to correspond to the position of the start frame selected by the start frame position selecting device.

6. The ultrasonic diagnostic apparatus according to claim 3, further comprising a start frame position image generating device for generating a start frame position image indicative of the position of the start frame which is input to the start frame position input device in slice images in a plurality of frames generated in chronological order by the image generating device,
wherein the display device displays the start frame position image generated by the start frame position image generating device.

7. The ultrasonic diagnostic apparatus according to claim 6, wherein the frame position image generating device generates the start frame position image by superimposing a start mark image indicating, by a mark, the position of the start frame which is input by the start frame position input device, on a time bar image indicative of a time base along which the image generating device has generated the slice images in the plurality of frames.

8. The ultrasonic diagnostic apparatus according to claim 1, further comprising an end frame selecting device for selecting an end frame at the time of finishing the chronological-order display of the slice images started by the display device in the slice images in the plurality of frames generated in the chronological order by the image generating device,
wherein the end frame selecting device selects, as the end frame, a frame generated by the image generating device at the time the injection time counting device finishes counting the time in which the contrast medium is injected into the subject, and
the display device sequentially displays the slice images of frames generated in the chronological order by the image generating device up to the slice image of the end frame selected by the end frame selecting device.

9. The ultrasonic diagnostic apparatus according to claim 8, further comprising an end frame change input device to which an instruction of changing the position of an end frame selected by the end frame selecting device is input by the operator,
wherein in the case where an instruction of changing the position of the end frame is input to the end frame change input device, the end frame selecting device selects the end frame so as to correspond to the instruction of changing the position of the end frame, which is input to the end frame change input device.

10. The ultrasonic diagnostic apparatus according to claim 7, further comprising an end frame position input device for inputting the position of the end frame in slice images in a plurality of frames generated in chronological order by the image generating device on the basis of an instruction from the operator,
wherein in the case where the position of the end frame is input by the end frame position input device, the end frame selecting device selects the end frame so as to correspond to the position of the end frame, which is input by the end frame position input device.

11. The ultrasonic diagnostic apparatus according to claim 10, wherein the position of the end frame in the chronological order in which the image generating device generates the slice images of the plurality of frames is input to the end frame position input device by the operator before a scan is performed by the scanning device.

12. The ultrasonic diagnostic apparatus according to claim 10, further comprising an end frame position selecting device, when a plurality of positions of the end frames are input by the end frame position input device, for selecting position of one of the plurality of end frames on the basis of an instruction from the operator,
wherein when the position of one of the end frames is selected by the end frame position selecting device, the end frame selecting device selects the end frame so as to correspond to the position of the end frame selected by the end frame position selecting device.

13. The ultrasonic diagnostic apparatus according to claim 10, further comprising an end frame position image generating device for generating an end frame position image indicative of the position of the end frame which is input to the end frame position input device in slice images in a plurality of frames generated in chronological order by the image generating device,
wherein the display device displays the end frame position image generated by the end frame position image generating device.

14. The ultrasonic diagnostic apparatus according to claim 13, wherein the end frame position image generating device generates the end frame position image by superimposing an end mark image indicating, by a mark, the position of the end frame which is input by the end frame position input device, on a time bar image indicative of a time base along which the image generating device has generated the slice images of the plurality of frames.

15. The ultrasonic diagnostic apparatus according to claim 1, wherein the image generating device generates at least a first moving picture and a second moving picture by generating a plurality of frames in chronological order of slice images of the subject on the basis of the echo signals obtained by the scanning device,
the start frame selecting device selects, as the start frames, a first start frame at the time the display device starts displaying the slice images in chronological order in the first moving picture generated by the image generating device and a second start frame at the time the display device starts displaying the slice images in chronological order in the second moving picture generated by the image generating device, and
the display device sequentially displays the slice image of the first start frame selected by the start frame selecting device in the first moving picture and subsequent slice images of frames generated in chronological order by the image generating device, and displays the slice image of the second start frame selected by the start frame selecting device in the second moving picture and subsequent slice images of frames generated in chronological order by the image generating device side by side so that the first start frame and the second start frame match on the time base.
